(19) United States
(12) Patent Application Publication
(10) Pub. No.: US 2025/0332007 A1
(43) Pub. Date: Oct. 30, 2025

(54) DIGIT FILLER WEARABLE GARMENT AND METHOD THEREOF

(71) Applicant: CATHY COKER, Indianapolis, IN (US)

(72) Inventor: CATHY COKER, Indianapolis, IN (US)

(21) Appl. No.: 19/195,188

(22) Filed: Apr. 30, 2025

Related U.S. Application Data
(60) Provisional application No. 63/640,224, filed on Apr. 30, 2024.

(51) Int. Cl. A61F 2/78; A41B 11/02; A61F 2/50; A61F 2/66

(52) U.S. Cl. CPC A61F 2/78; A41B 11/02; A61F 2002/5001; A61F 2002/6621

(57) ABSTRACT

A missing digit filler wearable garment apparatus generally configured to mimic a missing digit of a wearer. The wearable garment having at least one padding portion configured to mimic a missing toe or a missing finger of the wearer. The garment can include a first portion and second portion. The first portion can generally form a main body portion and extend from at or above a user's ankle to at least the mid-foot region of a user. The second portion can be formed to house on or more padding members that can mimic the amputated portion of the wearer.

### CROSS REFERENCE

This application is a U.S. non-provisional patent application and claims priority of U.S. provisional patent application Ser. No. 63/640,224, filed on Apr. 30, 2024, and incorporates by reference the subject matter thereof in its entirety.

### FIELD OF THE INVENTION

This invention relates generally to a new and improved digit filler wearable garment and method for use. In one aspect, the digit filler apparatus is configured to mimic a missing digit of a wearer.

### BACKGROUND

Most common items of footwear and handwear, such as socks and gloves, are made with a standard shape according to a person having all five digits in place. When a person has one or more digits amputated, there is an increased instability after amputation. The remaining digit or digits will often lean towards the missing digit causing a crowding and curvature of the forefoot. Patients who have had toe amputations tend to complain of migration of the remaining digits due to the destabilized digits. These patients may also experience an uncomfortable feeling when wearing regular shaped socks. The sock may feel loose at the place where the amputated toe used to be, and the sock may not fit well, migrate, and/or rotate when walking. Loosening of the sock may also occur and a wearer may need to tuck the sock under the remaining toes for a more secure and comfortable fit.

A regular sock used by a wearer who has a missing toe will not prevent migration of the remaining toes on the wearer's foot. Migration of the remaining toes will make the toes get closer and closer together, which will reduce the stability when standing and walking.

The currently available solution for this is a toe filler insert, which is a custom device that can be made from a mold of the wearer's foot, the device generally made specifically to fit one type of shoe. Currently the toe filler insert is made as part of the shoe as a whole. The toe filler is often difficult to mold because the digits are too small. Due to these difficulties, the toe filler insert is often made incorrectly, either too small or too big due to poor impression techniques and bulkiness of the material. The current materials used to make these toe fillers are high-dense materials, such as plastzole. The plastzole material can also cause skin irritation, wounds, and may cause further damage to the wearer's foot. The toe filler insert worn by the patient can rub onto the remaining toes, causing blisters and leading to infections. This also limits the ability for a user to switch between multiple shoes readily without the need of multiple customized shoes or shoe inserts specifically for each shoe.

Therefore, a need exists for an improved wearable garment apparatus that mimics a missing digit and helps stabilize the wearer's limbs with a natural feeling. Furthermore, it would be advantageous to have an apparatus that allows the wearer to use multiple shoes with comfort. Still further, it would be advantageous to have an apparatus molded directly from the wearer's foot and made of a soft material.

### BRIEF SUMMARY OF THE INVENTION

In one aspect, this disclosure is related a garment body having a first portion and a second portion, wherein the garment body includes a top surface a bottom surface a toe portion and heel portion. At least one padding portion can be contained in at least one of the toe portion or heel portion of the garment body, wherein the padding portion is formed to replace an amputated portion of a user. The padding portion provides support to the user when the garment apparatus is housed within a shoe to prevent unwanted friction. The padding portion further prevents or limits the migration of the remaining toes on the wearer's foot. A supporting band positioned or coupled between a first end of the second portion to allow a user to removably couple the padding portion. The second portion can include a flap closure to house the padding portion. The flap closure can include a silicone band for maintaining the position of the padding portion within the second portion. The second portion can fully encapsulate the padding portion to provide greater stability to the user and padding portion. A supporting member can extend from the second portion to the heel portion to provide further stability. A padded heel portion can be provided. The supporting member can extend along the bottom side of the first portion between the second portion and the heel portion.

In another aspect, this disclosure is related to a method of providing a wearable supporting device for a user with an amputation by first providing a user's foot for taking mold of the user's current digits to be amputated or a mold of the void remaining from the amputated portion of a user. A padded portion can then be generated based off the mold or one or more measurements of the user. A garment apparatus having a first portion and second portion can be provided. The first portion can primarily extend from above a user's ankle down to a mid-foot portion of the user. The padding portion can be positioned and secured within the second portion to provide stability and support to a user when the user's foot is inserted into a shoe. This garment allows a user to be able to more interchangeably select various shoes, while still maintain the same feel and support from any amputated portions.

In another aspect, this disclosure is related to a wearable garment having at least one padded portion configured to mimic a missing digit of the wearer.

The garment may comprise a sock having a padding configured to mimic a missing toe. The sock configured to give the user the impression that there is no missing toe, which may lead to better overall balance during gait.

The garment may comprise a glove having a padding configured to mimic a missing finger. The garment may be made of soft, flexible, breathable, washable, and reusable materials. The garment may be made of natural fibers, such as cotton or bamboo. The garment may be bespoken according to a specific need of the user.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 is a bottom view of an exemplary embodiment of an apparatus of the present disclosure for one amputated metatarsal of a user.

FIG. 2 is a side view of an exemplary embodiment of an apparatus of the present disclosure for metatarsal amputation of a user.

FIG. 3 is a bottom view of an exemplary embodiment of an apparatus of the present disclosure for two consecutive metatarsal amputations of a user.

FIG. 4 is a perspective view of an exemplary embodiment of an apparatus of the present disclosure for metatarsal amputations of a user.

FIG. 5 is a bottom view of an exemplary embodiment of an apparatus of the present disclosure for one digit amputation.

FIG. 6 is a partially exploded view of an exemplary embodiment of an apparatus of the present disclosure for one digit amputation.

FIG. 7A is a top view of an exemplary embodiment of an apparatus of the present disclosure with an interior filler portion encapsulated by the main body portion.

FIG. 7B is a bottom view of an exemplary embodiment of an apparatus of the present disclosure with an interior filler portion encapsulated by the main body portion.

FIG. 8 is a perspective view of an exemplary embodiment of an apparatus of the present disclosure for a transmetatarsal amputation or modular design.

FIG. 9 is a side view of an exemplary embodiment of an apparatus of the present disclosure for a transmetatarsal amputation or modular design.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit, or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

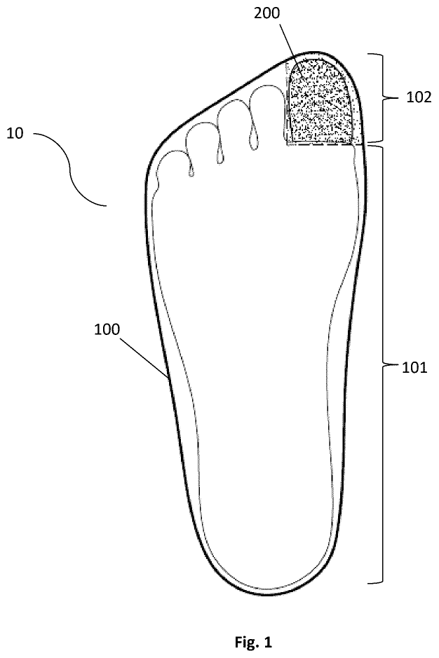

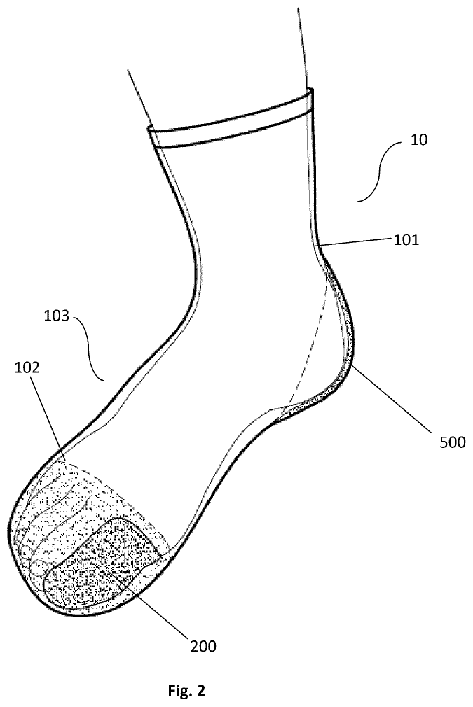

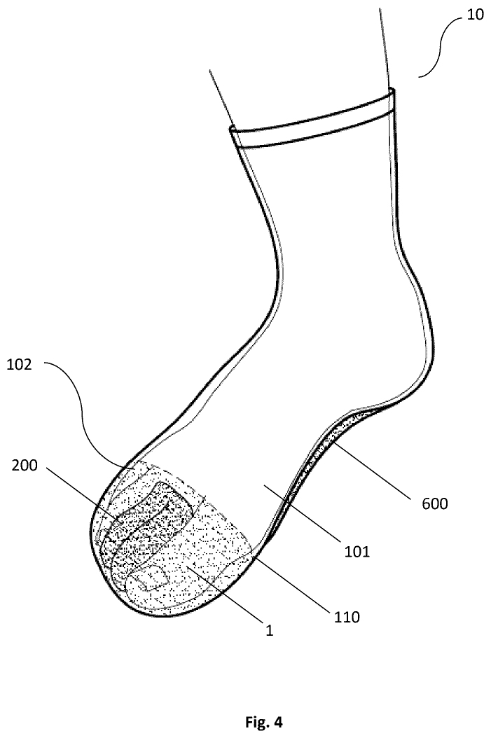

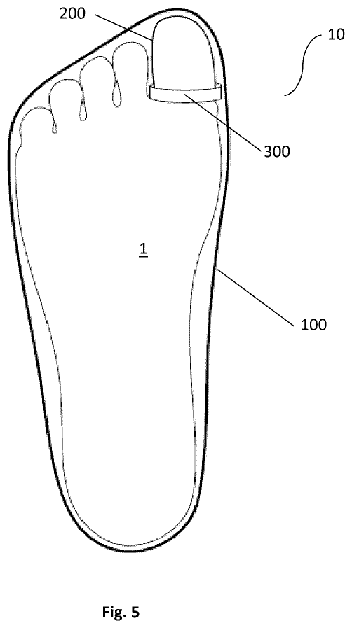

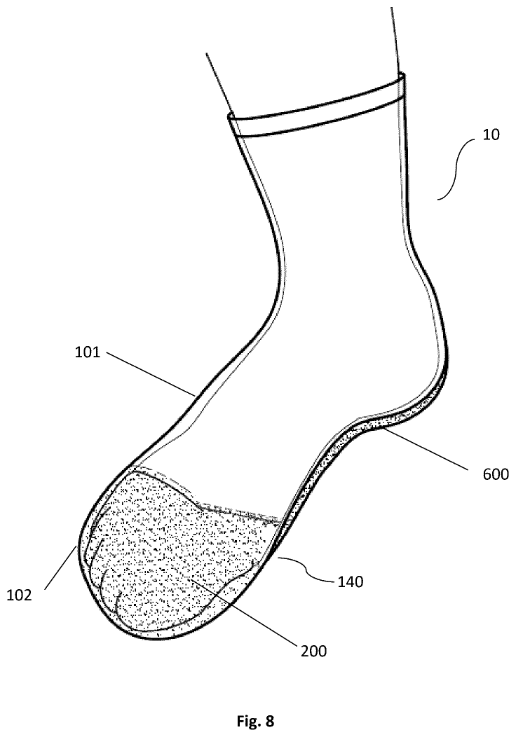

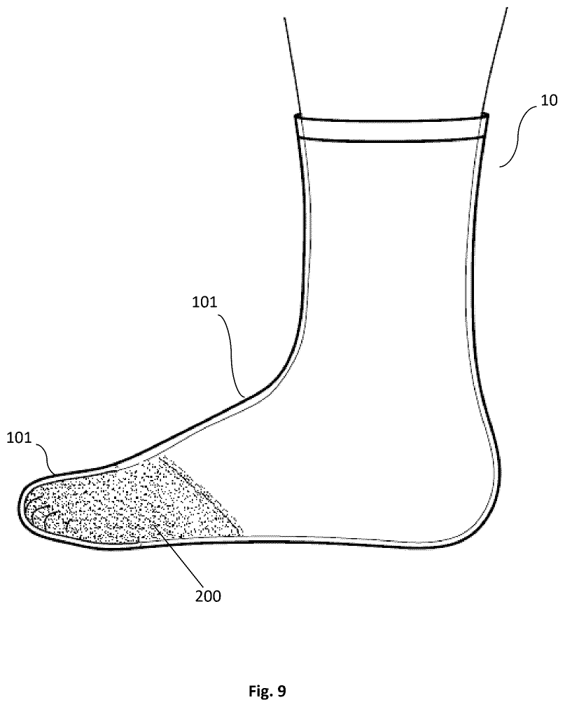

Referring now to FIGS. 1-9 of a wearable garment apparatus and method for use according to the present disclosure and generally referred to herein as apparatus 10. Most generally the apparatus 10 is generally an exterior shaped garment body 100 having at least one interior padding portion 200. In some exemplary embodiments, the garment 10 can have a first portion 1 and a second portion 1. In some exemplary embodiments, the first portion can be a main body portion 101 and a second portion can be a filler portion 102. The body portion 101 can generally be a sock or sleeve for a user to wear as shown in FIG. 1. In some exemplary embodiments, the main body portion 101 can be a longer compression leg sleeve with an enclosed foot portion 103 similar to a sock or stocking. In other embodiments, the main body portion 101 can generally be a garment portion that extends from above a user's ankle down the foot to about the transmetatarsal region of a user's foot as shown in FIGS. 8-9. In other exemplary embodiments, the main body portion 101 can extend to the toe region of a user as shown in FIG. 2. An apparatus 10 of the present disclosure can be a modular garment system providing a first portion 101 that can primarily form the main body of a garment 100, with a second portion 102 forming the toe or metatarsal region as shown in FIG. 4. The padding portion 200 can be used to fill the desired or necessary space needed for a user. In some exemplary embodiments, the padding material 200 may encompass the entire second portion 102 as shown in FIGS. 8-9. In other embodiments, only a predetermined amount of the second portion 102 may include padding material 200 as shown in FIG. 4.

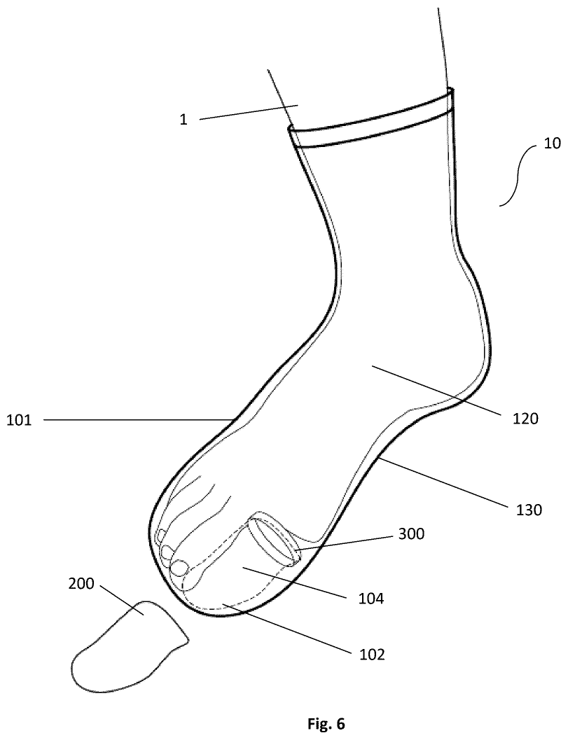

The filler portion 102 can then be coupled to the main body portion 101 to suit a user's specific needs. In some exemplary embodiments, the padded material can be removably coupled from the filler portion 102. The exterior shaped garment body 100 having generally a top surface 120 and a bottom surface 130 may have various shapes and sizes. The filler portion can then be coupled to an end portion 110 of the main garment portion 101. The filler portion 102 can have one or more cavities 104 configured to receive one or more interior padding members or portions 200 as shown in FIG. 6. The interior padding 200 may be sewed onto the body portion 101 or attached to the body portion 101 by other suitable means. In some exemplary embodiments, the padding portion can be comprised of a material that can be packed into the cavity, a semi-solid material, or a solid material to simulated a user's missing body portion.

The second portion 102 may be modular to allow for various preformed pockets 104 to be coupled to the second portion 102 to house the padding portion(s) 200. The at least one interior padding portion 200 can mimic a missing digit of a wearer who had a digit amputated. The one or more interior padding portions 200 can generally help stabilize and support the adjacent digits, rendering a more comfortable fitting of the garment 10 for patients who had digits amputations. The padding portion can further limit or inhibit migration of the other remaining digits of a user to ensure continuing stability. The padding portions 200 can similarly provide better support for a user 1 and fit within a shoe to prevent undesired wear or rubbing of a user's foot that may cause a user discomfort.

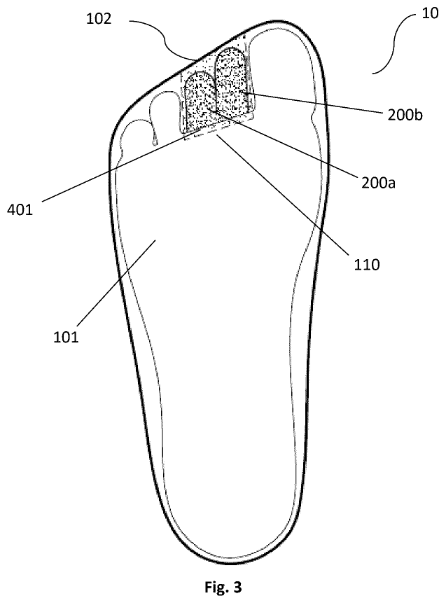

The interior padding member 200 may have various sizes and shapes and may be positioned within the garment 10 according to a pre-determined need of each wearer. In some exemplary embodiments, filler portion can have multiple padding members 200 as shown in FIG. 3. In some exemplary embodiments, the padding members 200a,b can be individualized or formed into a singular member to occupy the space of multiple missing digits. The padding 200 can be fully encapsulated within the filler portion 102 to provide greater stability of the padding. Similarly, the padding 200 can be formed from any suitable material to mimic the missing portion of a user's foot.

As shown in FIG. 8, a supporting band 300 can be further incorporated at the joint 104 between the main body portion 101 and the filler portion 102 and extend toward a heel portion of the main body portion to further support the filler portion's structure and juxtaposition. Additional one or more support straps can be incorporated into the main body portion 101 and filler portion 102. Similarly, support bands 300 can be extended between the filler portion 102 and the main body portion 101. This can further increase stability to the user as they are wearing and utilizing the apparatus of the present disclosure.

The interior padding 200 may have an adjustable size and shape and may be removably positioned within the garment 10, allowing for a universal garment configured to fit and fulfill different needs of different wearers. The removably padding members 200 can also be used and interchanged for different shoes that a wearer may have. The removable interior padding 200 may be secured within the second portion 102 pocket via a closure made of a flexible and durable materials, such as silicone or other materials. In some exemplary embodiments, the closure can be a sewn closure or hook and loop fasteners to maintain the padding within the second portion. In one exemplary embodiment, the flap closure can be comprised of a silicon band 300 can function as a closure as well. In one embodiment, the at least one interior padding 200 is configured to mimic a missing digit or portion of the wearer. In another embodiment, the at least one interior padding 200 is configured to mimic a missing finger of the wearer.

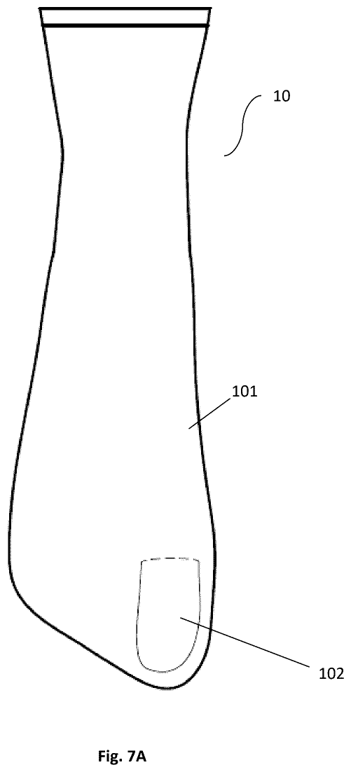

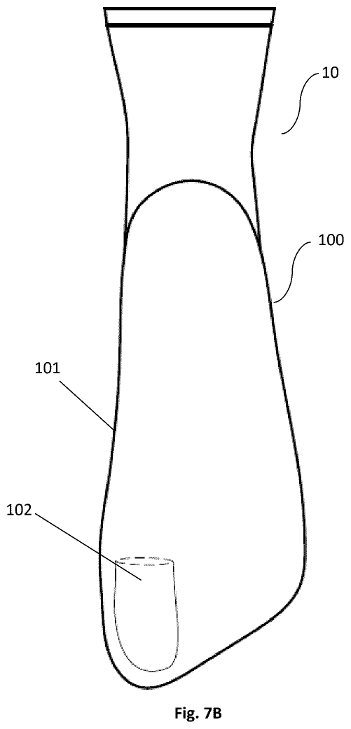

In yet another embodiment, the exterior shaped garment 100 and the at least one padding 200 may be made from a mold of the foot of the wearer. The at least one padding 200 may be made from an impression of the wearer's foot and the at least one padding material may be filled into the space of the at least one missing digit. The impression may be a cast, mold, image, digital capture, or other suitable methods. As shown in FIG. 7A-7B, the filler portion 102 can be internally located within the garment 100. The filler portion 102 can be completely surrounded by the main body portion to look uniform on the exterior surface of the garment surface 100.

For certain embodiments, including but not limited to transmetatarsal amputations or for tarsometatarsal amputations, the garment 10 may have a heel padding portion 500 and/or a supporting member 600 located underneath the garment and extending from the missing toes padding towards the heel padding 500, this will give an extra support along the plantar arch or bottom surface 130 and the heel to decrease the play and mobility in the forefoot due to the missing toes and to prevent the missing toes padding 200 to feel floppy (FIGS. 8 and 9). The supporting band/member 600 can be comprised of any suitable material, including but not limited to an elastic material an can run along the plantar portion of the foot on or proximate to the bottom surface 130 of the apparatus 10. The supporting band 600 can keep tension on the forefoot filler portion 140 during a user's gait. The exterior wearable garment 100 and the at least one interior padding 200 may be made of soft, flexible, breathable, washable, and reusable materials. The padding 200 may be made of soft foam material. In other embodiments the padding may be made of a hard foam. Alternatively, the padding 200 can be composed of a medical grade polymer material, such as a silicone or other material.

In yet other exemplary embodiments, the wearable garment comprises compression stockings, including but not limited to, thigh length, below the knee, foot compression, or combinations thereof, having either the fitted or removable toe filler. In yet other exemplary embodiments, the wearable garment comprises ankle socks, crew socks, or no-show socks, having either the fitted or removable toe filler.

The present disclosure further provides a method of providing a patient with a wearable garment for additional support for amputated digits from a user's foot. A patient can have a mold made of the portion to be amputated or alternatively of the void left after the amputation. A padding portion can then be generated or made based upon the mold of the user. A garment having a first portion and second portion can be provided. The first portion can be generally the main body portion. In some exemplary embodiments, the main body portion can extend from above the ankle or heel and down to a mid-foot region of a user. The padding portion can then be positioned or housed within the second portion of the garment. The garment can help provide stability and support to a user when the foot is positioned within a shoe.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A wearable garment apparatus, comprising:
a garment body having a first portion and a second portion, wherein the garment body includes a top surface a bottom surface a toe portion and heel portion; and
at least one padding portion contained in at least one of the toe portion or heel portion of the garment body, wherein the padding portion is formed to replace an amputated portion of a user.

2. The wearable garment apparatus of claim 1, wherein the padding portion provides support to the user when the garment apparatus is housed within a shoe to prevent unwanted friction.

3. The wearable garment apparatus of claim 1, further comprising a supporting band positioned at a first end of the second portion to allow a user to removably couple the padding portion.

4. The wearable garment apparatus of claim 1, further comprising a flap closure, wherein the flap closure is a silicone band configured to maintain the padding portion within the second portion.

5. The wearable garment apparatus of claim 4, wherein the second portion fully encapsulates the padding portion to provide greater stability to the padding portion.

6. The wearable garment apparatus of claim 5, further comprising a supporting member extending from the second portion to the heel portion to provide further stability and support to the padding portion, wherein the supporting member provides tension on a forefoot portion containing the padding portion during a user's gait.

7. The wearable garment apparatus of claim 6, further comprising a padded heel portion.

8. The wearable garment apparatus of claim 7, wherein the supporting member extends along the bottom side of the first portion between the second portion and the heel portion.

9. The wearable garment apparatus of claim 8, wherein the supporting band is incorporated at a joint between the first portion and the second portion.

10. The wearable garment apparatus of claim 9, wherein the first portion is a main body portion of the garment and the second portion is the filler portion positioned at the toe portion end of the garment.

11. The wearable garment apparatus of claim 10, wherein the filler portion is molded from a user's foot prior to amputation.

12. The wearable garment apparatus of claim 1, wherein the wearable garment is a sock having at least one padding portion to mimic a missing toe of the user.

13. A method of providing a support device for a user amputation comprising:
providing a user's foot for taking a mold of the current user's digits to be amputated or a mold of the void remaining from the amputated portion of a user;
providing a padding portion from the mold or one or more measurements of the user;
providing a garment having a first portion and a second portion, wherein the first portion is a primarily extends from above a user's ankle down to a mid-foot portion of a user;
wherein the padding portion is positioned within the second portion to provide stability and support to a user when the user's foot is inserted into a shoe.

14. The method of claim 13, wherein the garment further includes a padded heel portion.

15. The method of claim 14, wherein a strap portion extends between the first end of the second portion and the help portion to provider additional support to the padding portion within the second portion.
